(19) United States
(12) Patent Application Publication
(10) Pub. No.: US 2005/0188477 A1
(43) Pub. Date: Sep. 1, 2005

(54) Composition for dyeing keratin fibers, comprising at least one alcohol oxidase and at least one silicone chosen from amino silicones and oxyalkylenated silicones, and process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001, US

(21) Appl. No.: 11/044,079

(22) Filed: Jan. 28, 2005

Related U.S. Application Data
(60) Provisional application No. 60/545,485, filed on Feb. 19, 2004.

(30) Foreign Application Priority Data
Jan. 28, 2004 (FR) 0400782

(51) Int. Cl. A61K 7/13

(52) U.S. Cl. 8/405

(57) ABSTRACT

A composition for dyeing keratin fibers, for example, human keratin fibers such as hair, comprising, in a medium that is suitable for dyeing, at least one oxidation dye precursor, at least one alcohol oxidase enzyme, at least one substrate for the at least one alcohol oxidase enzyme and at least one silicone chosen from amino silicones and oxyalkylenated silicones; process for dyeing keratin fibers comprising applying the composition, and also a dyeing device or “kit”.

This application claims benefit of U.S. Provisional Application No. 60/545,485, filed Feb. 19, 2004 and French Patent Application No. FR 0400782 filed Jan. 28, 2004, which are both herein incorporated by reference.

Disclosed herein is a composition for dyeing keratin fibers, for example, human keratin fibers, such as hair, comprising, in a medium that is suitable for dyeing, at least one oxidation dye precursor, at least one alcohol oxidase enzyme, at least one substrate for the at least one alcohol oxidase enzyme, and at least one silicone chosen from amino silicones and oxyalkylenated silicones.

It is known practice to dye keratin fibers, for example, human hair, with dye compositions comprising oxidation dye precursors, such as ortho- or para-phenylenediamines, ortho- or para-aminophenols, and heterocyclic compounds, which are generally referred to as oxidation bases. These oxidation bases, may be colorless or weakly colored compounds which, when combined with oxidizing products, may give rise to colored compounds by a process of oxidative condensation.

It is also known that the shades obtained with these oxidation bases may be varied by combining them with couplers or coloration modifiers. The couplers or coloration modifiers may be chosen, for example, from aromatic meta-diamines, meta-aminophenols, meta-diphenols, and certain heterocyclic compounds, such as indole compounds.

The variety of molecules used as oxidation bases and couplers can make it possible to obtain a wide range of colors.

The “permanent” coloration obtained by means of these oxidation dyes usually satisfy a certain number of requirements. For example, it should have no toxicological drawbacks and it may allow shades of the desired intensity to be obtained and may have good resistance to external agents such as light, bad weather, washing, permanent waving, perspiration and rubbing.

The dyes may also allow white hairs to be covered and, further, they may be as unselective as possible, that is to say that they may allow the smallest possible differences in coloration to be produced over the entire length of the same keratin fiber, which is generally differently sensitized (i.e. damaged) between its end and its root.

The dyeing may be performed in a strongly alkaline medium, in the presence of hydrogen peroxide. The use of alkaline media, however, in the presence of hydrogen peroxide may have the drawback of causing considerable degradation of the fibers, and also bleaching of keratin fibers, which may not always desirable.

The oxidation dyeing of keratin fibers may also be performed using oxidizing systems other than hydrogen peroxide, such as enzymatic systems. For example, Patent Application No. FR 2 769 219 describes the use of a uricase enzyme and of its uric acid substrate in oxidation dyeing to dye keratin fibers. Patent Application No. EP-A-0 310 675 describes the use of an oxidation dye precursor of benzenic type in combination with enzymes such as pyranose oxidase or glucose oxidase. Further, for example, Patent Application No. FR 2 833 492 describes the use of the enzyme alcohol oxidase as a sole enzyme in an oxidation dye composition for dyeing keratin fibers.

At least one embodiment of the present inventor is to provide novel stable compositions for dyeing keratin fibers by oxidation dyeing, using an oxidizing system other than hydrogen peroxide.

The inventor has discovered that it is possible to achieve this by using at least one oxidation dye precursor, at least one alcohol oxidase enzyme, at least one substrate for the at least one alcohol oxidase enzyme, and at least one silicone chosen from amino silicones and oxyalkylenated silicones, in a composition for dyeing keratin fibers, for example, human keratin fibers, such as hair.

The compositions disclosed herein may have at least one of the following advantages: leading to the production of strong, unselective and fast colors, generating varied shades of intense and uniform color, without significant degradation of the hair, improving the hold of permanent-waved hair, and reducing the porosity of the hair.

Other characteristics, aspects, subjects and advantages of the compositions disclosed herein will emerge even more clearly on reading the description and the examples that follow.

It is recalled that, in accordance with what is generally accepted, the term “silicone” means any organosilicon polymer or oligomer of linear or cyclic, branched or crosslinked structure, of variable molecular weight, obtained by polymerization and/or polycondensation of suitably functionalized silanes, and comprising a repetition of main units in which the silicon atoms are linked together via oxygen atoms (siloxane bond —Si—O—Si—), optionally substituted hydrocarbon-based radicals, being linked directly via a carbon atom on the silicon atoms. Examples of hydrocarbon-based radicals include alkyl radicals, for example, C₁-C₁₀ alkyls, and, further, for example, methyl, fluoroalkyl radicals, wherein the alkyl portion is C₁-C₁₀, and aryl radicals, such as phenyl.

As used herein, the term “amino silicone” means any silicone comprising at least one functional group chosen from primary, secondary, and tertiary amine and quaternary ammonium functional groups.

As used herein, the term “polyoxyalkylenated silicone” means any silicone comprising at least one oxyalkylenated group of the type (—CₓH₂ₓO—), wherein x ranges from 2 to 6, and a is greater than or equal to 2.

The compositions disclosed herein comprise at least one silicone chosen from amino silicones and oxyalkylenated silicones. In some embodiments, the compositions disclosed herein comprise a mixture of several amino silicones or several oxyalkylenated silicones, or alternatively of several amino silicones and oxyalkylenated silicones.

For example, the amino silicones may be chosen from:
(a) compounds corresponding to the following formula: 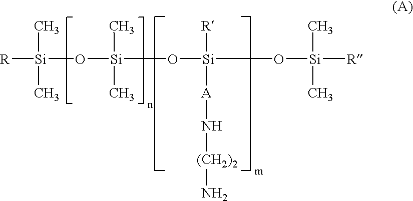
wherein:
R, R′ and R″, which may be identical or different, are each chosen from C₁-C₄ alkyl radicals, for example, CH₃; C₁-C₄ alkoxy radicals, for example, methoxy; and OH;
A is chosen from linear and branched, C₃-C₈ alkylene radicals and, for example, C₃-C₆ alkylene radicals; and
m and n are each integers dependent on the molecular weight and whose sum ranges from 1 to 2000.

In one embodiment, R, R′ and R″, which may be identical or different, are each chosen from C₁-C₄ alkyl and hydroxyl radicals, A is a C₃ alkylene radical and m and n are such that the weight-average molecular mass of the compound ranges from 5000 to 500 000. Compounds of this type are referred to in the CTFA dictionary as “amodimethicones”.

In another embodiment, R, R′ and R″, which may be identical or different, are each chosen from C₁-C₄ alkoxy and hydroxyl radicals, wherein at least one of the radicals R or R″ is an alkoxy radical and A is a C₃ alkylene radical. The hydroxyl/alkoxy molar ratio may, for example, range from 0.2/1 to 0.4/1 and, further, for example, equal to 0.3/1. Moreover, m and n are such that the weight-average molecular mass of the compound ranges from 2000 to 106. For example, n is a number ranging from 0 to 999 and m is a number ranging from 1 to 1000, wherein the sum of n and m ranges from 1 to 1000.

An example of a compound in this category is the product Belsil® ADM 652 sold by Wacker.

In another embodiment, R and R″, which are different, are chosen from C₁-C₄ alkoxy and hydroxyl radicals, wherein at least one of the radicals R or R″ is an alkoxy radical, R′ is a methyl radical and A is a C₃ alkylene radical. The hydroxyl/alkoxy molar ratio may, for example, range from 1/0.8 to 1/1.1 and, for example, may be equal to 1/0.95. Moreover, m and n can be such that the weight-average molecular mass of the compound ranges from 2000 to 200 000. For example, n is a number ranging from 0 to 999 and m is a number ranging from 1 to 1000, wherein the sum of n and m ranges from 1 to 1000.

For example, mention may be made of the product Fluid WR® 1300 sold by the company Wacker.

In another embodiment, R and R″ are each a hydroxyl radical, R′ is a methyl radical and A is chosen from C₄-C₈ alkylene radicals, for example, A can be a C₄ alkylene radical. Moreover, m and n are such that the weight-average molecular mass of the compound ranges from 2000 to 10⁶. For example, n ranges from 0 to 1999 and m ranges from 1 to 2000, wherein the sum of n and m ranges from 1 to 2000.

A product of this type is sold under the name DC 28299 by Dow Corning.

It should be noted that the molecular mass of these silicones is determined by gel permeation chromatography (room temperature, polystyrene standards; styragem μ columns; THF eluent; a flow rate of 1 mm/m; 200 μl of a 0.5% by weight solution of silicone in THF are injected and detection is performed by refractometry and UV-metry).
(b) compounds corresponding to formula (B) below:
   (R¹)ₐ(T)₃₋ₐSi[OSi(T)₂]ₙ—[OSi(T)_{b}(R¹)_{2-b}]ₘ—OSi(T)₃₋ₐ-(R¹)ₐ (B)
   wherein:

T chosen from a hydrogen atom and phenyl, hydroxyl (—OH), and C₁-C₈ alkyl radicals, for example, a methyl radical;

a is the number 0 or an integer ranging from 1 to 3. In one embodiment, a is the number 0;

b is 0 or 1, and, in one embodiment, b is 1;

m and n are numbers such that the sum (n+m) can range, for example, from 1 to 2000 and, further, for example, from 50 to 150, n can be a number ranging from 0 to 1999 and, for example, from 49 to 149, and m can be a number ranging from 1 to 2000 and, for example, from 1 to 10;

R¹ is a monovalent radical of formula —C_{q}H_{2q}L wherein q is a number ranging from 2 to 8 and L is an optionally quaternized amino group chosen from the following groups:
—N(R²)—CH₂—CH₂—N(R²)₂,
—N(R²)₂,
—N^{⊕}(R²)₃Q⁻,
—N^{⊕}(R²)(H)₂Q⁻,
—N^{⊕}(R²)₂HQ⁻, and
—N(R²)—CH₂—CH₂—N^{⊕}(R²)(H)₂Q⁻,
wherein R² is chosen from a hydrogen atom, a phenyl, a benzyl, and a saturated monovalent hydrocarbon-based radical, for example, a C₁-C₂₀ alkyl radical, and Q⁻ is chosen from halide ions, such as, fluoride, chloride, bromide, and iodide.

A product corresponding to this definition is the polymer known in the CTFA dictionary as “trimethylsilyl amodimethicone”, corresponding to the formula (C) below: 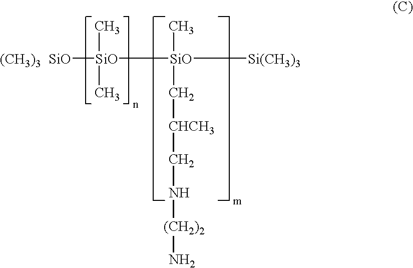
wherein m and n are numbers such that the sum (n+m) can range, for example, from 1 to 2000 and, further, for example, from 50 to 150, n can be a number ranging from 0 to 1999 and, for example, from 49 to 149, and m can be a number ranging from 1 to 2000 and, for example, from 1 to 10.

Such compounds are described, for example, in Patent Application No. EP-A-95238; a compound of formula (C) is sold, for example, under the name Q2-8220 by the company OSI.
(c) compounds corresponding to formula (D) below: 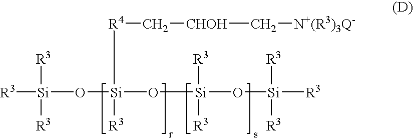
   wherein:

R³ is chosen from monovalent C₁-C₁₈ hydrocarbon-based radicals, and, for example, C₁-C₁₈ alkyl and C₂-C₁₈ alkenyl radicals, for example, a methyl radical;

R⁴ is chosen from divalent hydrocarbon-based radicals, for example, C₁-C₁₈ alkylene radicals and divalent C₁-C₁₈, and for example C₁-C₈, alkylenoxy radicals;

Q⁻ is chosen from halide ions, for example, Q⁻ may be a chloride ion;

r represents an average statistical value from 2 to 20 and, for example, from 2 to 8; and

s represents an average statistical value from 20 to 200 and, for example, from 20 to 50.

Such compounds are described, for example, in Patent No. U.S. Pat. No. 4,185,087.

An example of a compound falling within this class is the product sold by the company Union Carbide under the name “Ucar Silicone ALE 56”.

When the compounds of formula (D) are used, one embodiment is their joint use with cationic and/or nonionic surfactants.

For example, it is possible to use the product sold under the name “Cationic Emulsion DC 929” by the company Dow Corning, which comprises, besides amodimethicone, a cationic surfactant comprising a mixture of products corresponding to the formula: 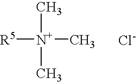
wherein R⁵ is chosen from C₁₄-C₂₂ alkenyl and alkyl radicals derived from tallow fatty acids, and known under the CTFA name “tallowtrimonium chloride”, in combination with a nonionic surfactant of formula:

C₉H₁₉—C₆H₄—(OC₂H₄)₁₀—OH, known under the CTFA name as “Nonoxynol 10”.

Use may be made, for example, of the product sold under the name “Cationic Emulsion DC 939” by the company Dow Corning, which comprises, besides amodimethicone, a cationic surfactant which is trimethylcetylammonium chloride and a nonionic surfactant of formula: C₁₃H₂₇—(OC₂H₄)₁₂—OH, known under the CTFA name “trideceth-12”.

Another commercial product that may be used in the compositions disclosed herein is the product sold under the name “Dow Corning Q2 7224” by the company Dow Corning, comprising, in combination, the trimethylsilyl amodimethicone of formula (C) described above, a nonionic surfactant of formula: C₈H₁₇—C₆H₄—(OCH₂CH₂)₄₀—OH, known under the CTFA name “octoxynol-40”, and a second nonionic surfactant of formula: C₁₂H₂₅—(OCH₂-CH₂)₆—OH, known under the CTFA name “isolaureth-6”, and propylene glycol.

The oxyalkylenated silicones are chosen from compounds of general formula (I), (II), (III), (IV) and (V): 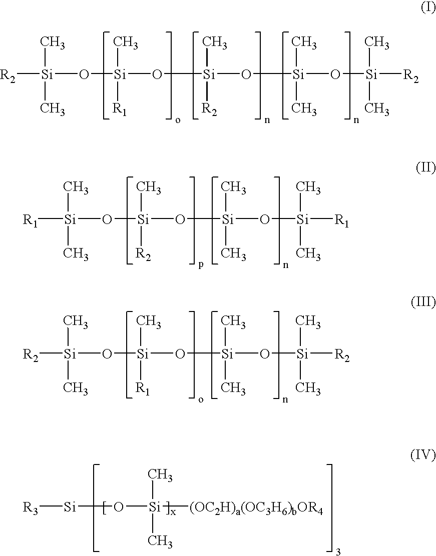
wherein:
R₁, which may be identical or different, is chosen from linear and branched C₁-C₃₀ alkyl and phenyl radicals;
R₂, which may be identical or different, is chosen from —C_{c}H_{2c}—O—(C₂H₄O)ₐ(C₃H₆O)_{b}—R₅ and —C_{c}H_{2c}—O—(C₄H₈O)ₐ—R₅;
R₃ and R₄, which may be identical or different, is chosen from linear and branched C₁ to C₁₂ alkyl radicals and, for example, a methyl radical;
R₅, which may be identical or different, is chosen from a hydrogen atom, linear and branched alkyl radicals comprising from 1 to 12 carbon atoms, linear and branched alkoxy radicals comprising from 1 to 6 carbon atoms, linear and branched acyl radicals comprising from 2 to 30 carbon atoms, a hydroxyl radical, —SO₃M, C₁-C₆ aminoalkoxy radicals optionally substituted on the amine, C₂-C₆ aminoacyl radicals optionally substituted on the amine, —NHCH₂CH₂COOM, —N(CH₂CH₂COOM)₂, aminoalkyl radicals optionally substituted on the amine and on the alkyl chain, C₂-C₃₀ carboxyacyl radicals, a group optionally substituted with one or two, identical or different, substituted aminoalkyl radicals, —CO(CH₂)_{d}COOM, —COCHR₇(CH₂)_{d}COOM, —NHCO(CH₂)_{d}OH, —NH₃Y, and a phosphate group;
M, which may be identical or different, is chosen from a hydrogen atom, Na, K, Li, NH₄. and organic amines;
R₇ is chosen from a hydrogen atom and radical —SO₃M;
d is a number ranging from 1 to 10;
m is a number ranging from 0 to 20;
n is a number ranging from 0 to 500;
o is a number ranging from 0 to 20;
p is a number ranging from 1 to 50;
a is a number ranging from 0 to 50;
b is a number ranging from 0 to 50;
a+b is a number greater than or equal to 2;
c is a number ranging from 0 to 4;
x is a number ranging from 1 to 100; and
Y is chosen from monovalent mineral and organic anions, such as halides (chloride and bromide) and sulfates and carboxylates (acetate, lactate, and citrate),
with the proviso that when the silicone is of formula (II) and R₅ is a hydrogen atom, then n is greater than 12.

Such silicones are sold, for example, by the company Goldschmidt under the trade names Abil WE 09, Abil EM 90, Abil B8852, Abil B8851, Abil B8843 and Abil B8842, by the company Dow Corning under the names Fluid DC 190, DC3225 C, Q2-5220, Q25354 and Q2-5200, by the company Rhone-Poulenc under the names Silbione Oil 70646 and Rhodorsil Oil 10634, by the company General Electric under the names SF1066 and SF1188, by the company SWS Silicones under the name Silicone Copolymer F 754, by the company Amerchol under the name Silsoft Beauty Aid SL, by the company Shin-Etsu under the name KF 351, by the company Wacker under the name Belsil DMC 6038, by the company Siltech under the names Silwax WD-C, Silwax WD-B, Silwax WD-IS, Silwax WSL, Silwax DCA 100 and Siltech Amine 65, by the company Fanning Corporation under the names Fancorsil SLA and Fancorsil LIM1, and by the company Phoenix under the name Pecosil.

These silicones are described, for example, in Patent Nos. U.S. Pat. No. 5,070,171, U.S. Pat. No. 5,149,765, U.S. Pat. No. 5,093,452 and U.S. Pat. No. 5,091,493.

In one embodiment, polyoxyalkylenated silicones corresponding to the general formula (II) or (III) are used. For example, these formulae satisfy at least one, and further, for example, all, of the following conditions:
c is equal to 2 or 3;
R₁ is a methyl radical;
R₅ is chosen from a methyl radical, C₁₂-C₂₂ acyl radicals, and CO(CH₂)_{d}COOM;
a is a number ranging from 2 to 25 and, for example, from 2 to 15;
b is equal to 0;
n is a number ranging from 0 to 100; and
p is a number ranging from 1 to 20.

The polyoxyalkylenated silicones disclosed herein may also be chosen from the silicones of formula (V) below:
([Z(R₂SiO)_{q}R′₂SiZO][(CₙH₂ₙO)ᵣ])ₛ (V)
wherein:
R₂ and R′₂, which may be identical or different, are each chosen from monovalent C₁-C₃₀ hydrocarbon-based radicals;
n is an integer ranging from 2 to 4;
q is a number greater than or equal to 4, for example, ranging from 4 to 200 and, even further, for example, from 4 to 100;
r is a number greater than or equal to 4, for example, ranging from 4 to 200 and, even further, for example, from 5 to 100;
s is a number greater than or equal to 4, for example, ranging from 4 to 1000 and, further, for example, from 5 to 300;
Z is a divalent organic group linked to the adjacent silicon atom via a carbon-silicon bond and to the polyoxyalkylene block (CnH₂nO) via an oxygen atom,
the average molecular weight of each siloxane block ranges from 400 to 10 000, the average molecular weight of each polyoxyalkylene block ranges from 300 to 10 000;
the siloxane blocks represent from 10% to 95% by weight of the block copolymer,
the number-average molecular weight of the block copolymer can range from 2500 to 1 000 000, for example, from 3000 to 200 000 and, even further, for example, from 6000 to 100 000.

R₂ and R′₂, which may be identical or different, are each chosen from groups comprising at least one radical chosen from linear and branched alkyl radicals, for example, methyl, ethyl, propyl, butyl, pentyl, hexyl, octyl, decyl and dodecyl radicals, aryl radicals, such as phenyl and naphthyl, aralkyl, and alkylaryl radicals, such as benzyl and phenylethyl, and tolyl and xylyl radicals.

Z, in one embodiment, is chosen from —R″—, —R″—CO—, —R″—NHCO—, —R″—NH—CO—CO—NH—R″′—, and —R″—OCONH—R′″—NHCO—, wherein R″ is chosen from linear and branched C₁-C₆ divalent alkylene groups, for example, ethylene, propylene, and linear and branched butylene groups, and R″′ is chosen from divalent alkylene groups and divalent arylene groups, for example, —C₆H₄—, —C₆H₄—C₆H₄—, —C₆H₄—CH₂—C₆H₄—, and —C₆H₄—C(CH₃)₂C₆H₄—.

In another embodiment, Z is chosen from divalent alkylene radicals, for example, linear and branched —C₃H₆— radicals and a —C₄H₈— radical.

The preparation of the block copolymers used in the compositions disclosed herein is described in European Patent Application No. EP 0 492 657 A1, the teaching of which is included in the present description.

Such products are sold, for example, under the name Silicone Fluid FZ-2172 by the company OSI.

The at least one silicone used in the compositions disclosed herein may be in the form of aqueous solutions or optionally in the form of aqueous dispersions or emulsions.

The at least one silicone may be present in an amount ranging from 0.05% to 30% by weight and, for example, from 0.1% to 15% by weight, relative to the total weight of the composition.

The at least one alcohol oxidase enzyme that may be used in the dye compositions disclosed herein belong to the category EC 1.1.3 of the enzyme nomenclature (see Enzyme Nomenclature, Academic Press Inc., 1992).

The at least one alcohol oxidase enzyme may be chosen, for example, from primary alcohol oxidases (EC 1.1.3.13), secondary alcohol oxidases (EC 1.1.3.18), long hydrocarbon-based chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7), also known as aryl alcohol oxidases.

In one embodiment, the at least one alcohol enzyme used in the compositions disclosed herein is a primary alcohol oxidase (EC 1.1.3.13).

In another embodiment, the at least one alcohol oxidase enzyme forms a particular category of 2-electron oxidoreductase enzymes.

The at least one alcohol oxidase enzyme used in the dye compositions disclosed herein may be derived from a plant, animal or microorganism (bacterium, fungus, yeast, microalga or virus) extract, from differentiated or undifferentiated cells, obtained in vivo or in vitro, which are genetically modified or unmodified, or synthetic (obtained via chemical or biotechnological synthesis).

In one embodiment, the at least one alcohol oxidase enzyme may be derived from one of the following species: Rhodococcus erythropolis and Pseudomonas pseudoalcaligenes, which are bacteria, Aspergillus niger, Kamagataella pastoris, Phanerochaete chrysosporium, Polyporus obtusus, Hansenula polymorpha, Poria contigua, Penicillium simplicissimum and Pleurotus pulmonarius (fungi), Pichia sp. (pastoris, methanolica, angusta) and Candida sp. (boidinii, albicans, tropicalis) (yeasts), Pinus strobus, which is a species of plant origin, and Gastropode mollusc and Manduca Sexta, which are of animal origin.

In one embodiment, the at least one alcohol oxidase enzyme used in the compositions disclosed herein is an alcohol oxidase enzyme derived from Pichia pastoris.

The at least one alcohol oxidase enzyme may be present in the dye compositions disclosed herein in an amount ranging from 0.05% to 20% by weight, for example, from 0.1% to 10% by weight and, further, for example, from 0.5% to 8% by weight, relative to the total weight of the composition.

The enzymatic activity of the at least one alcohol oxidase enzyme may be defined from the oxidation of the donor under aerobic conditions. The unit U corresponds to the amount of enzyme leading to the generation of 1 μmol of hydrogen peroxide per minute at a given pH and at a temperature of 25° C.

For example, the amount of the at least one alcohol oxidase enzyme can range from 10³ U to 10⁵ U, and, for example, from 2×10³ U to 5×10⁴ U per 100 g of dye composition.

The at least one substrate for the at least one alcohol oxidase enzyme is also known as donor(s) for the enzyme.

The at least one substrate for the at least one alcohol oxidase enzyme may, for example, be an alcohol chosen from primary and secondary alcohols, long-hydrocarbon-based-chain alcohols and aromatic alcohols. For example, mention may be made, as donors for the primary alcohol oxidases, of primary alcohols comprising from 1 to 6 carbon atoms; as donors for the aryl alcohol oxidases: benzyl alcohol, 4-tert-butylbenzyl alcohol, 3-hydroxy-4-methoxybenzyl alcohol, veratryl alcohol, 4-methoxybenzyl alcohol, and cinnamic alcohol; 2,4-hexadiene-1-ol may also be used as donors for the aryl alcohol oxidases.

In another embodiment, the at least one substrate for the at least one alcohol oxidase enzyme is a compound bearing at least one functional group chosen from aliphatic and aromatic alcohol functional groups, suitable for reaction with the enzyme used. The compound bearing at least one functional group chosen from aliphatic and aromatic alcohol functional groups may, for example, be an oxidation dye precursor or a cosmetically acceptable adjuvant, for example a polymer, a surfactant or a preserving agent bearing at least one alcohol functional group. In one embodiment, the at least one substrate for the at least one alcohol oxidase enzyme is an oxidation dye precursor bearing at least one functional group chosen from aliphatic and aromatic alcohol functional groups. For example, N-(β-hydroxypropyl)-para-phenylenediamine, which bears a primary alcohol functional group, may serve as an oxidation base and as the at least one substrate for the at least one alcohol oxidase enzyme. Similarly, oxidation couplers, such as meta- or para-aminophenol, may serve the two functions. Such precursors are described hereinbelow. In this embodiment, the use of other substrates for the at least one alcohol enzyme is optional.

Thus, the compositions disclosed herein are compositions for dyeing keratin fibers, for example, human keratin fibers, such as hair, comprising, in a medium that is suitable for dyeing, at least the following compounds: at least one oxidation dye precursor; at least one alcohol oxidase enzyme; at least one substrate, bearing at least one alcohol functional group, for the at least one alcohol oxidase enzyme, and at least one silicone chosen from amino silicones and oxyalkylenated silicones, wherein the at least one substrate is optionally substituted (i.e. replaced) totally or partially with the at least one oxidation dye precursor in the case where the at least one oxidation dye precursor bears at least one functional group chosen from aliphatic and aromatic alcohol functional groups.

In one embodiment, the at least one substrate for the at least one alcohol oxidase enzyme may be present in an amount ranging, for example, from 0.01% to 60% by weight, relative to the total weight of the composition, and, further, for example, from 0.05% to 30% by weight, relative to the total weight of the composition.

The at least one oxidation dye precursor may be an oxidation base chosen, for example, from para-phenylenediamines, bis(phenyl)alkylenediamines, para-amino-phenols, ortho-aminophenols, heterocyclic bases, and addition salts thereof.

Examples of the para-phenylenediamines include para-phenylenediamine, para-tolylenediamine, 2-chloro-para-phenylenediamine, 2,3-dimethyl-para-phenylene-diamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methyl-aniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-,N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and acid addition salts thereof.

In one embodiment, the para-phenylenediamines mentioned above that can be used in the compositions disclosed herein include para-phenylenediamine, para-tolylenediamine, 2-isopropyl-para-phenylenediamine, 2-β-hydroxyethyl-para-phenylene-diamine, 2-β-hydroxyethyloxy-para-phenylenediamine, 2,6-dimethyl-para-phenylene-diamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and acid addition salts thereof.

Examples of the bis(phenyl)alkylenediamines include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetra-methylenediamine, N,N′-bis(β-hydroxyethyl)-N,N′-bis(4-aminophenyl)tetra-methylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and acid addition salts thereof.

Examples of the para-aminophenols include para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6-[((5′-amino-2′-hydroxy-3′-methyl)phenyl)methyl]-2-methylphenol, bis(5′-amino-2′-hydroxy)phenylmethane, and acid addition salts thereof.

Examples of the ortho-aminophenols include 2-aminophenol, 2-amino-5-methylphenol, 2-amino-6-methylphenol, 5-acetamido-2-aminophenol, and acid addition salts.

Examples of the heterocyclic bases include pyridine derivatives, pyrimidine derivatives, and pyrazole derivatives.

Examples of the pyridine derivatives include the compounds described, for example, in Patent Nos. GB 1 026 978 and GB 1 153 196, such as 2,5-diaminopyridine, 2-(4-methoxyphenyl)amino-3-aminopyridine, and 3,4-diaminopyridine, and acid addition salts thereof.

Other examples of pyridine oxidation bases that may be used in the compositions disclosed herein include the 3-aminopyrazolo[1,5-a]pyridine oxidation bases and addition salts thereof described, for example, in Patent Application No. FR 2 801 308, such as pyrazolo[1,5-a]pyrid-3-ylamine; 2-acetylaminopyrazolo-[1,5-a]pyrid-3-ylamine; 2-morphol in-4-ylpyrazolo[1,5-a]pyrid-3-ylamine; 3-aminopyrazolo[1,5-a]pyrid-2-carboxylic acid; 2-methoxypyrazolo[1,5-a]pyrid-3-ylamino; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-yl-pyrazolo[1,5-a]pyrid-3-ylamine; pyrazolo[1,5-a]pyridine-3,5-diamine; 5-morpholin-4-yl-pyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol; and acid addition salts thereof.

Examples of the pyrimidine derivatives include the compounds described, for example, in Patent Nos. DE 2 359 399; JP 88-169 571; JP 05 631 124; EP 0 770 375 and Patent Application No. WO 96/15765, such as 2,4,5,6-tetraaminopyrimidine, 4-hydroxy-2,5,6-triaminopyrimidine 2-hydroxy-4,5,6-triaminopyrimidine, 2,4-dihydroxy-5,6-diamino-pyrimidine, 2,5,6-triaminopyrimidine, and addition salts thereof, and tautomeric forms thereof, when a tautomeric equilibrium exists.

Examples of the pyrazole derivatives include the compounds described in Patent Nos. DE 3 843 892, DE 4 133 957 and Patent Application Nos. WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino- 1,3-dimethyl-5-hydrazinopyrazole, 1-benzyl-4,5-diamino-3-methyl pyrazole, 4,5-diamino-3-tert-butyl-1-methylpyrazole, 4,5-diamino-1-tert-butyl-3-methylpyrazole, 4,5-diamino-1-(β-hydroxyethyl )-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl )amino-1-methylpyrazole, and acid addition salts thereof.

When the at least one oxidation dye precursor is an oxidation base it may be present in an amount ranging, for example, from 0.0001% to 20% and, further, for example, from 0.005% to 6% by weight, relative to the total weight of the composition.

The at least one oxidation dye precursor may be a standard oxidation coupler chosen, for example, from meta-phenylenediamines, meta-aminophenols, meta-diphenols, naphthalene-based couplers, heterocyclic couplers, and addition salts thereof.

Examples of standard oxidation couplers include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(P-hydroxyethyl)amino-3,4-methylenedioxybenzene, 2,6-bis(β-hydroxyethylamino)toluene, and addition salts thereof.

When the at least one oxidation dye precursor is an oxidation coupler it may be present in an amount ranging, for example, from 0.0001% to 20% by weight, and further, for example, from 0.005% to 6% by weight, relative to the total weight of the composition.

The acid addition salts that may be used for the oxidation bases and couplers may be chosen, for example, from the hydrochlorides, hydrobromides, sulfates, citrates, succinates, tartrates, lactates, tosylates, benzenesulfonates, phosphates, and acetates.

The addition salts that may be used in the compositions disclosed herein may be chosen, for example, from addition salts with sodium hydroxide, potassium hydroxide, ammonia, amines, and alkanolamines.

In some embodiments, the dye compositions disclosed herein may further comprise at least one direct dye, which may be chosen, for example, from neutral, acidic and cationic nitrobenzene dyes; neutral, acidic and cationic azo direct dyes; neutral, acidic and cationic quinone, for example, anthraquinone direct dyes; azine, methine, azomethine, triarylmethane and indoamine direct dyes; and natural direct dyes. In one embodiment, the compositions disclosed herein may, for example, comprise at least one direct dye chosen from cationic direct dyes and natural direct dyes.

Examples of the cationic direct dyes that may be used include cationic azo direct dyes described in Patent Application Nos. WO 95/15144, WO 95/01772 and EP-714 954.

Among these compounds, mention may be made, for example, of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methylphenylhydrazono)methyl]pyridinium methyl sulfate.

Examples of the natural direct dyes that may be used include lawsone, juglone, alizarin, purpurin, carminic acid, kermesic acid, purpurogallin, proto-catechaldehyde, indigo, isatin, curcumin, spinulosin, and apigenidin. It is also possible to use extracts or decoctions comprising these natural dyes, for example, henna-based poultices or extracts.

The at least one direct dye may be present in an amount ranging, for example, from 0.001% to 20% by weight, and, further, for example, from 0.005% to 10% by weight, relative to the total weight of the composition.

The dye compositions disclosed herein may also comprise at least one additional adjuvant. The at least one additional adjuvant may be chosen from adjuvants conventionally used in hair dye compositions; for example, antioxidants, penetrating agents, sequestering agents, fragrances, buffers, dispersants, surfactants, conditioning agents, such as additional volatile and non-volatile, modified and unmodified silicones other than the at least one silicone chosen from amino and oxyalkylenated silicones used in the compositions disclosed herein; cationic polymers, cations, film-forming agents, ceramides, preserving agents, opacifiers, vitamins, and provitamins.

The at least one adjuvant may be present in an amount ranging from 0.01% to 20% by weight, relative to the weight of the composition.

Needless to say, a person skilled in the art will take care to select this or these optional additional compound(s) such that the advantageous properties intrinsically associated with the oxidation dye compositions disclosed herein are not, or are not substantially, adversely affected by the envisaged addition(s).

The medium that is suitable for dyeing, also known as the dye support, generally comprises water or a mixture of water and at least one organic solvent to dissolve the compounds that would not be sufficiently water-soluble. The at least one organic solvent may, for example, be where appropriate, chosen from enzyme substrates, such as ethanol and isopropanol. The at least one organic solvent may also be chosen from non-substrate enzyme compounds such as polyol ethers, for example, 2-butoxyethanol, propylene glycol, propylene glycol monomethyl ether, diethylene glycol monomethyl ether, monoethyl ether, and phenoxyethanol.

The at least one organic solvent may be present in an amount ranging from 1% to 40% by weight, relative to the total weight of the composition, and, further, for example, from 5% to 30% by weight, relative to the total weight of the composition.

The pH of the compositions disclosed herein ranges, for example, from 6 to 11 and, further, for example, from 7 to 10. It may be adjusted for the desired value using at least one agent chosen from acidifying and basifying agents usually used in the dyeing of keratin fibers, or alternatively using standard buffer systems.

The acidifying agents may be chosen from mineral and organic acids, for example, hydrochloric acid; orthophosphoric acid; sulfuric acid; carboxylic acids, such as acetic acid, tartaric acid, citric acid, and lactic acid; and sulfonic acids.

The basifying agents may be chosen, for example, from aqueous ammonia, alkyl metal carbonates, alkanolamines such as monoethanolamine, diethanolamine and triethanolamine and also derivatives thereof, sodium hydroxide, potassium hydroxide and the compounds of formula (III) below: 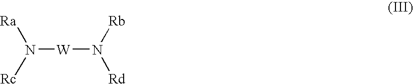
wherein: W is a propylene residue optionally substituted with at least one group chosen from a hydroxyl group and C₁-C₄ alkyl groups; Ra, Rb, Rc and Rd, which may be identical or different, are each chosen from a hydrogen atom, C₁-C₄ alkyl, and C₁-C₄ hydroxyalkyl radicals.

The dye composition may be in various forms, such as in a form chosen from liquids, creams, gels, and any other forms that are suitable for dyeing keratin fibers, such as human hair.

When the at least one oxidation dye precusor and the at least one alcohol oxidase enzyme are present in the same composition, the composition is, for example, free of oxygen gas, so as to avoid any premature oxidation of the at least one oxidation dye precursor.

Further disclosed herein is a process for dyeing keratin fibers, for example, human keratin fibers, such as hair, comprising applying, to the fibers, at least one composition disclosed herein and leaving the at least one dye composition on the fibers for a time period that is sufficient to develop the desired coloration.

The color is then revealed by bringing together the at least one alcohol oxidase enzyme and the at least one substrate in the presence of oxygen.

The at least one composition is applied to the keratin fibers. After leaving the at least one composition to act, for example, from 3 to 60 minutes, and, further, for example, from 5 to 40 minutes, the keratin fibers are rinsed, washed with shampoo, rinsed again, and then dried.

When the at least one composition is a composition in ready-to-use form, it comprises, in a medium that is suitable for dyeing keratin fibers, at least one oxidation dye precursor, at least one alcohol oxidase enzyme, at least one substrate for the at least one alocohol oxidase enzyme, and at least one silicone chosen from amino silicones and oxyalkylenated silicones, and wherein the at least one composition is then stored in anaerobic form, free of oxygen gas.

According to one embodiment, the process further comprises, before applying the at least one composition to the keratin fibers, separately storing, at least one composition (A) comprising, in a medium that is suitable for dyeing keratin fibers, at least one oxidation dye precursor, and, at least one composition (B) comprising, in a medium that is suitable for dyeing keratin fibers, at least one alcohol oxidase enzyme, wherein the at least one composition (A) and/or the at least one composition (B) comprises at least one substrate for the at least one alcohol oxidase enzyme, and at least one composition (A) and/or at least one composition (B) comprises at least one silicone chosen from amino silicones and oxyalkylenated silicones; and mixing compositions (A) and (B) at the time of use.

In another embodiment, the process further comprises, before applying the at least one composition, separately storing, on the one hand, at least one composition (A) comprising, in a medium that is suitable for dyeing keratin fibers, at least one oxidation dye precursor, at least one substrate for the at least one alcohol oxidase enzyme and at least one silicone chosen from amino silicones and oxyalkylenated silicones and at least one composition (B) comprising, in a medium that is suitable for dyeing keratin fibers, at least one alcohol oxidase enzyme, and then in mixing the compositions (A) and (B) at the time of use.

The color may be developed at acidic, neutral or alkaline pH. In the case where the process is performed using the at least one composition (A) comprising at least one oxidation dye precursor, at least one substrate for the at least one alcohol oxidase enzyme and at least one silicone chosen from amino silicones and oxyalkylenated silicones and at least one composition (B) comprising at least one alcohol oxidase enzyme, the at least one alcohol oxidase enzyme may be added to the at least one composition just at the time of use, or may be used starting with a composition comprising it, which is applied simultaneously with or sequentially to the at least one composition.

The at least one composition (B) (referred to as the oxidizing composition) may also comprise at least one adjuvant conventionally used in hair dye compositions and as defined previously.

The pH of the at least one “oxidizing” composition B is such that, after mixing with the dye composition A, the pH of the resulting composition applied to the keratin fibers ranges, for example, from 6 to 11 and, further, for example, from 7 to 10. It may be adjusted to the desired value using at least one agent chosen from acidifying and basifying agents usually used in the dyeing of keratin fibers and as defined previously.

In one embodiment, the compositions disclosed herein are applied at a temperature ranging, for example, from room temperature to 220° C. and, further, for example, from room temperature to 60° C.

Further disclosed herein is a multi-compartment device or dyeing “kit”, wherein at least one first compartment comprises the at least one composition (A) as defined above and at least one second compartment comprises the at least one composition (B) as defined above. This device may be equipped with a means for applying the desired mixture to the hair, such as the devices described in Patent No. FR 2 586 913.

The examples that follow serve to illustrate the various embodiments disclosed herein without, however, being limiting in nature.

### EXAMPLES

The following compositions are prepared:

| Constituent | Comp. 1 | | Comp. 2 | | Comp. 3 | | Comp. 4 | | Comp. 5 | |
|---|---|---|---|---|---|---|---|---|---|---|
| Silwax WD IS | 0.3 | g | | | | | | | | |
| Silphos A 100 | | | 0.5 | g | | | | | | |
| Silwet L77 | | | | | 0.4 | g | | | | |
| Dow Corning 939 | | | | | | | 1.2 | g | | |
| Emulsion | | | | | | | | | | |
| Dow Corning Q2- | | | | | | | | | 1 | g |
| 8220 Fluid | | | | | | | | | | |
| Ethanol | 25 | g | 25 | g | 25 | g | 25 | g | 25 | g |
| para-Phenylene- | 3 × 10⁻³ | mol | 3 × 10⁻³ | mol | 3 × 10⁻³ | mol | 3 × 10⁻³ | mol | 3 × 10⁻³ | mol |
| diamine | | | | | | | | | | |
| meta-Aminophenol | 3 × 10⁻³ | mol | 3 × 10⁻³ | mol | 3 × 10⁻³ | mol | 3 × 10⁻³ | mol | 3 × 10⁻³ | mol |
| Alcohol oxidase | 20000 | units | 20000 | units | 20000 | units | 20000 | units | 20000 | units |
| 2-amino-2-methyl-1- | pH 7 | | pH 7 | | pH 7 | | pH 7 | | pH 7 | |
| propanol qs | | | | | | | | | | |
| Distilled water qs | 100 | g | 100 | g | 100 | g | 100 | g | 100 | g |

Dow Corning 939 Emulsion: polydimethylsiloxane containing aminoethylaminopropyl groups, at a concentration of 35% in water.

Dow Corning Q2-8220 Fluid: mixture of polydimethylsiloxane containing aminoethylaminoisobutyl groups/polydimethylsiloxane.

The alcohol oxidase used was that sold by the company Biozyme Laboratories, in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above compositions were applied to locks of natural and permanent-waved grey hair containing 90% white hairs, and left to act for 30 minutes. The bath ratio was set at 5. The alcohol oxidase was added extemporaneously. The hair was then rinsed, washed with a standard shampoo and then dried.

The hair was dyed in khaki-green shades.

1. A composition for dyeing keratin fibers, comprising, in a medium that is suitable for dyeing:
at least one oxidation dye precursor;
at least one alcohol oxidase enzyme;
at least one substrate, bearing at least one alcohol functional group, for the at least one alcohol oxidase enzyme, and
at least one silicone chosen from amino silicones and oxyalkylenated silicones,
wherein the at least one substrate is optionally substituted totally or partially with the at least one oxidation dye precursor in the case where the at least one oxidation dye precursor bears at least one alcohol functional group chosen from aliphatic and aromatic alcohol functional groups.

2. The composition according to claim 1, wherein the keratin fibers are human keratin fibers.

3. The composition according to claim 2, wherein the human keratin fibers are hair.

4. The composition according to claim 1, wherein the at least one silicone is chosen from amino silicones corresponding to formula (A) below: 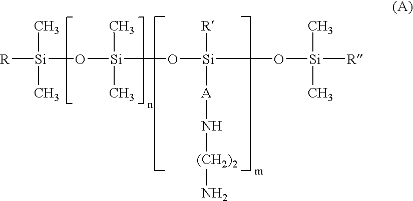
wherein:
R, R′ and R″, which may be identical or different, are each chosen from C₁-C₄ alkyl radicals, C₁-C₄ alkoxy radicals, and OH;
A is chosen from linear and branched C₃-C₈ alkylene radicals; and
m and n are integers dependent on the molecular weight and whose sum ranges from 1 to 2000.

5. The composition according to claim 1, wherein the at least one silicone is chosen from amino silicones corresponding to formula (B) below:
(R¹)ₐ(T)₃₋ₐSi[OSi(T)₂]ₙ—[OSi(T)_{b}(R¹)_{2-b}]ₘ—OSi(T)₃₋ₐ-(R¹)ₐ
wherein:
T is chosen from a hydrogen atom and phenyl, hydroxyl, and C₁-C₈ alkyl radicals;
a is the number 0 or an integer ranging from 1 to 3;
b is 0 or 1;
m and n are numbers such that the sum (n+m) ranges from 1 to 2000;
wherein n is a number ranging from 0 to 1999, and m is a number ranging from 1 to 2000; and
R¹ is a monovalent radical of formula —C_{q}H_{2q}L, wherein q is a number ranging from 2 to 8 and L is an optionally quaternized amino group chosen from the following groups:
—N(R²)—CH₂—CH₂—N(R²)₂;
—N(R²)₂;
—N^{⊕}(R²)₃Q⁻;
—N^{⊕}(R²)(H)₂Q⁻;
—N^{⊕}(R²)₂HQ⁻; and
—N(R²)—CH₂—CH₂—N^{⊕}(R²)(H)₂Q⁻;
wherein R² is chosen from a hydrogen atom, a phenyl, a benzyl, and saturated monovalent hydrocarbon-based radicals and Q⁻ is chosen from halide ions.

6. The composition according to claim 5, wherein m and n are numbers such that the sum (n+m) ranges from 50 to 150.

7. The composition according to claim 5, wherein n is a number ranging from 49 to 149.

8. The composition according to claim 5, wherein m is a number ranging from 1 to 10.

9. The composition according to claim 1, wherein the at least one silicone chosen from amino silicones is a “trimethylsilyl amodimethicone” corresponding to formula (C) below: 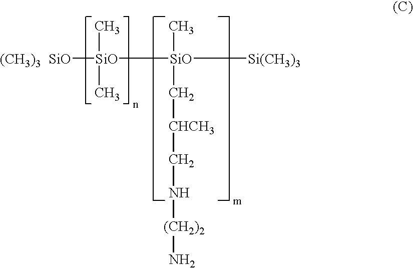
wherein:
m and n are numbers such that the sum (n+m) ranges from 1 to 2000;
wherein n is a number ranging from 0 to 1999, and m is a number ranging from 1 to 2000

10. The composition according to claim 1, wherein the at least one silicone is chosen from amino silicones corresponding to formula D below: 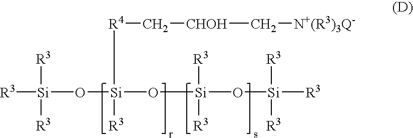
wherein:
R³ is chosen from monovalent C₁-C₁₈ hydrocarbon-based radicals;
R⁴ is chosen from divalent hydrocarbon-based radicals;
Q⁻ is chosen from halide ions;
r represents an average statistical value ranging from 2 to 20; and
s represents an average statistical value ranging from 20 to 200.

11. The composition according to claim 10, wherein R⁴ is chosen from C₁-C₁₈ alkylene radicals and divalent C₁-C₁₈ alkylenoxy radicals.

12. The composition according to claim 10, wherein r represents an average statistical value ranging from 20 to 50.

13. The composition according to claim 10, wherein r represents an average statistical value ranging from 2 to 8.

14. The composition according to claim 1, wherein the at least one silicone is an oxyalkylenated silicone chosen from the compounds of formula (I), (II), (III), (IV) and (V): 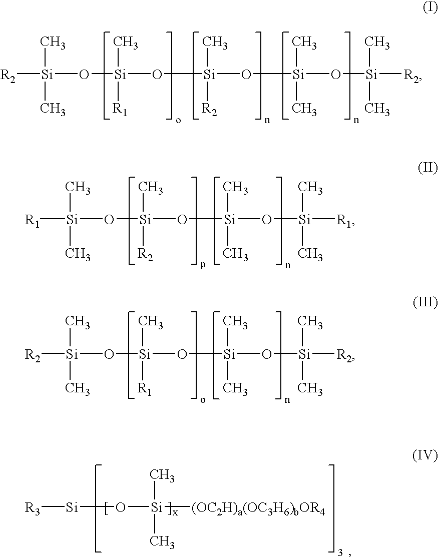
wherein:
R₁, which may be identical or different, is chosen from linear and branched C₁-C₃₀ alkyl and phenyl radicals;
R₂, which may be identical or different, is chosen from —C_{c}H_{2c}—O—(C₂H₄O)ₐ(C₃H₆O)_{b}—R₅ radicals and —C_{c}H_{2c}—O—(C₄H₈O)ₐ—R₅ radicals;
R₃ and R₄, which may be identical or different, is chosen from linear and branched C₁ to C₁₂ alkyl radicals;
R₅, which may be identical or different, is chosen from a hydrogen atom, linear and branched alkyl radicals comprising from 1 to 12 carbon atoms, linear and branched alkoxy radicals comprising from 1 to 6 carbon atoms, linear and branched acyl radicals comprising from 2 to 30 carbon atoms, a hydroxyl radical, —SO₃M, C₁-C₆ aminoalkoxy radicals optionally substituted on the amine, C₂-C₆ aminoacyl radicals optionally substituted on the amine, —NHCH₂CH₂COOM, —N(CH₂CH₂COOM)₂, aminoalkyl radicals optionally substituted on the amine and on the alkyl chain, C₂-C₃₀ carboxyacyl radicals, a group optionally substituted with one or two, identical or different, substituted aminoalkyl radicals, —CO(CH₂)_{d}COOM, —COCHR₇(CH₂)_{d}COOM, —NHCO(CH₂)_{d}OH, —NH₃Y, and a phosphate group;
M, which may be identical or different, is chosen from a hydrogen atom, Na, K, Li, NH₄, and organic amines;
R₇ is chosen from a hydrogen atom and radical —SO₃M;
d is a number ranging from 1 to 10;
m is a number ranging from 0 to 20;
n is a number ranging from 0 to 500;
o is a number ranging from 0 to 20;
p is a number ranging from 1 to 50;
a is a number ranging from 0 to 50;
b is a number ranging from 0 to 50;
a+b is a number greater than or equal to 2;
c is a number ranging from 0 to 4;
x is a number ranging from 1 to 100;
Y is chosen from monovalent mineral and organic anions; with the proviso that when the at least one silicone is of formula (II) and R₅ is a hydrogen atom, then n is greater than 12, and
([Z(R₂SiO)_{q}R′₂SiZO][CₙH₂ₙO)ᵣ])ₛ (V)
wherein:
R₂ and R′₂, which may be identical or different, are each chosen from monovalent C₁-C₃₀ hydrocarbon-based radicals;
n is an integer ranging from 2 to 4;
q is a number greater than or equal to 4;
r is a number greater than or equal to 4;
s is a number greater than or equal to 4;
Z is a divalent organic group linked to the adjacent silicon atom via a carbon-silicon bond and to the polyoxyalkylene block (CₙH₂ₙO) via an oxygen atom; and
wherein the average molecular weight of each siloxane block ranges from 400 to 10 000, the average molecular weight of each polyoxyalkylene block ranges from 300 to 10 000, the siloxane blocks represent from 10% to 95% by weight of the block copolymer, and the number-average molecular weight of the block copolymer ranges from 2500 to 1 000 000.

15. The composition according to claim 14, wherein Y is a halide ion chosen from chloride and bromide or a sulfate or carboxylate ion chosen from acetate, lactate, and citrate.

16. The composition according to claim 14, wherein q is a number ranging from 4 to 200.

17. The composition according to claim 16, wherein q is a number ranging from 4 to 100.

18. The composition according to claim 14, wherein r is a number ranging from 4 to 200.

19. The composition according to claim 18, wherein r is a number ranging from 5 to 100.

20. The composition according to claim 14, wherein s is a number ranging from 4 to 1000.

21. The composition according to claim 20, wherein s is a number ranging from 5 to 300.

22. The composition according to claim 1, wherein the composition comprises a mixture chosen from mixtures of at least two amino silicones, mixtures of at least two oxyalkylenated silicones, and mixtures of at least one amino silicone and at least one oxyalkylenated silicone.

23. The composition according to claim 1, wherein the at least one silicone is present in an amount ranging from 0.05% to 30% by weight, relative to the total weight of the composition.

24. The composition according to claim 23, wherein the at least one silicone is present in an amount ranging from 0.1% to 15% by weight, relative to the total weight of the composition.

25. The composition according to claim 1, wherein the at least one alcohol oxidase enzyme is chosen from primary alcohol oxidases (EC 1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7).

26. The composition according to claim 25, wherein the at least one alcohol oxidase enzyme is derived from one of the following species: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp. (pastoris, methanolica, angusta) and Candida sp. (boidinii, albicans, tropicalis), Pinus strobus, Gastropode mollusc, and Manduca sexta.

27. The composition according to claim 26, wherein the at least one alcohol oxidase enzyme is derived from Pichia pastoris.

28. The composition according to claim 1, wherein the at least one alcohol oxidase enzyme is present in an amount ranging from 0.05% to 20% by weight, relative to the total weight of the composition.

29. The composition according to claim 28, wherein the at least one alcohol oxidase enzyme is present in an amount ranging from 0.1% to 10% by weight, relative to the total weight of the composition.

30. The composition according to claim 29, wherein the at least one alcohol oxidase enzyme is present in an amount ranging from 0.5% to 8% by weight, relative to the total weight of the composition.

31. The composition according to claim 1, wherein the at least one alcohol oxidase enzyme is present in an amount ranging from 10³ U to 10⁵ U per 100 g of the dye composition.

32. The composition according to claim 31, wherein the at least one alcohol oxidase enzyme is present in an amount ranging from 2×10³ U to 5×10⁴U per 100 g of dye composition.

33. The composition according to claim 1, wherein the at least one substrate for the at least one alcohol oxidase enzyme is an alcohol chosen from primary and secondary alcohols, long-hydrocarbon-chain alcohols, and aromatic alcohols.

34. The composition according to claim 33, wherein the at least one substrate for the at least one alcohol oxidase enzyme is present in an amount ranging from 0.01% to 60% by weight, relative to the total weight of the composition.

35. The composition according to claim 34, wherein the at least one substrate for the at least one alcohol oxidase enzyme is present in an amount ranging from 0.05% to 30% by weight, relative to the total weight of the composition.

36. The composition according to claim 1, wherein the at least one oxidation dye precursor is an oxidation base chosen from para-phenylenediamines, bis(phenyl)alkylene-diamines, para-aminophenols, ortho-aminophenols, heterocyclic bases, and addition salts thereof.

37. The composition according to claim 36, wherein the at least one oxidation dye precursor is present in an amount ranging from 0.0001% to 20% by weight, relative to the total weight of the composition.

38. The composition according to claim 1, wherein the at least one oxidation dye precursor is an oxidation coupler chosen from meta-phenylenediamines, meta-aminophenols, meta-diphenols, naphthalene-based couplers, heterocyclic couplers, and addition salts thereof.

39. The composition according to claim 38, wherein the at least one oxidation dye precursor is present in an amount ranging from 0.0001% to 20% by weight, relative to the total weight of the composition.

40. The composition according to claim 1, further comprising at least one direct dye chosen from natural and cationic direct dyes.

41. A process for dyeing keratin fibers comprising,
applying to the fibers at least one composition comprising, in a medium that is suitable for dyeing,
at least one oxidation dye precursor;
at least one alcohol oxidase enzyme;
at least one substrate, bearing at least one alcohol functional group, for the at least one alcohol oxidase enzyme; and
at least one silicone chosen from amino silicones and oxyalkylenated silicones;
wherein the at least one substrate is optionally substituted totally or partially with the at least one oxidation dye precursor in the case where the at least one oxidation dye precursor bears at least one alcohol functional group chosen from aliphatic and aromatic alcohol functional groups and
leaving the at least one composition on the fibers for a time period sufficient to develop the desired coloration.

42. The process according to claim 41, wherein the keratin fibers are human keratin fibers.

43. The process according to claim 41, wherein the human keratin fibers are hair.

44. The process according to claim 41, wherein the at least one composition is a ready-to-use composition stored in anaerobic form, free of oxygen gas.

45. The process according to claim 41, further comprising, before applying the at least one composition to the keratin fibers,
separately storing,
at least one composition (A) comprising, in a medium that is suitable for dyeing, at least one oxidation dye precursor and
at least one composition (B) comprising, in a medium that is suitable for dyeing, at least one alcohol oxidase enzyme,
wherein the at least one composition (A) and/or the at least one composition (B) comprises at least one substrate for the at least one alcohol oxidase enzyme, and the at least one composition (A) and/or the at least one composition (B) comprises at least one silicone chosen from amino silicones and oxyalkylenated silicone; and
mixing together the compositions (A) and (B) at the time of use.

46. The process according to claim 41, further comprising, before applying the at least one composition,
separately storing
at least one composition (A) comprising, in a medium that is suitable for dyeing, at least one oxidation dye precursor, at least one substrate for the at least one alcohol oxidase enzyme, and at least one silicone chosen from amino silicones and oxyalkylenated silicones and
at least one composition (B) comprising, in a medium that is suitable for keratin fibers, at least one alcohol oxidase enzyme, and
mixing compositions (A) and (B) at the time of use.

47. A multi-compartment device or dyeing “kit” comprising,
at least one first compartment comprising at least one composition (A) comprising, in a medium that is suitable for dyeing, at least one oxidation dye precursor, and
at least one second compartment comprising at least one composition (B) comprising, in a medium that is suitable for dyeing, at least one alcohol oxidase enzyme,
wherein the at least one composition (A) and/or the at least one composition (B) comprises at least one substrate for the at least one alcohol oxidase enzyme, and the at least one composition (A) and/or the at least one composition (B) comprises at least one silicone chosen from amino silicones and oxyalkylenated silicone.

48. A multi-compartment device or dyeing “kit” comprising,
at least one first compartment comprising at least one composition (A) comprising, in a medium that is suitable for dyeing, at least one oxidation dye precursor, at least one substrate for the at least one alcohol oxidase enzyme, and at least one silicone chosen from amino silicones and oxyalkylenated silicones and
at least one second compartment comprising at least one composition (B) comprising, in a medium that is suitable for keratin fibers, at least one alcohol oxidase enzyme.
